(19) United States
(12) Patent Application Publication
(10) Pub. No.: US 2021/0057062 A1
(43) Pub. Date: Feb. 25, 2021

(54) COMPUTER SYSTEM AND METHOD FOR CONCURRENT CLINICAL DOCUMENT IMPROVEMENT AND CODING

(71) Applicant: 3M Innovative Properties Company, Saint Paul, MN (US)

(72) Inventors: Julie A. Salomon, Atlanta, GA (US); Jeffrey S. Seese, Hanover, PA (US); Diana M. Ortiz, Wausau, WI (US); Julie L. Imburgia, Draper, UT (US); Christine F. Bayless, St. George, UT (US)

(73) Assignee: 3M Innovative Properties Company, Saint Paul, MN (US)

(21) Appl. No.: 16/999,235

(22) Filed: Aug. 21, 2020

Related U.S. Application Data
(60) Provisional application No. 62/890,698, filed on Aug. 23, 2019.

(51) Int. Cl. G16H 10/60; G06Q 10/10

(52) U.S. Cl. CPC G16H 10/60; G06Q 10/10

(57) ABSTRACT

A computer system implements a concurrent Clinical Document Improvement (CDI) and coding workflow, such as may be used to generate codesets for use in connection with medical bills. The concurrent workflow enables steps in the coding process to be performed close to admission and concurrently with (e.g., interwoven with) the patient visit and steps in the CDI process, thereby increasing the accuracy of the resulting codesets and the efficiency of the codeset generation process. In particular, this concurrent coding workflow may be used to resolve DRG mismatches and enable codesets to be finalized immediately upon discharge of the patient from the hospital.

### BACKGROUND

Medical coding involves generating codes that may be used to justify medical bills based on documentation of healthcare services that were provided to patients. For example, a coder may receive a written diagnosis of a patient and use the diagnosis to generate one or more codes that represent the diagnosis. For every injury, diagnosis, and medical procedure, there is a corresponding code. A variety of coding standards exist, such as various versions of International Classification of Diseases (ICD) codes and Current Procedure Terminology (CPT) codes. Generating codes that are accurate and complete based on a patient encounter typically is a tedious and time-consuming process. Often a complete set of codes for a patient encounter is not generated until after the patient has been discharged. This results both in a delay in billing and difficulties in confirming the accuracy of codes which could be avoided if the codes were ready for review while the patient and healthcare providers were still readily available.

Clinical documentation improvement (CDI), also known as “clinical documentation integrity”, refers to the best practices, processes, technology, people, and joint effort between healthcare providers and billing coders that advocates the completeness, precision, and validity of provider documentation inherent to billing code sets (e.g. ICD-10-CM, ICD-10-PCS, CPT, HCPCS), as sanctioned by the Health Insurance Portability and Accountability Act (HIPAA) in the United States. CDI professionals typically act as intermediaries between inpatient coders, who translate diagnoses into data, and healthcare providers and nurses. Because many clinical coders do not have patient care backgrounds, and because healthcare providers may not realize the importance of or take the time required to produce accurate documentation, the CDI professional serves to make the connection between these two groups. Various software exists for assisting in the CDI process.

The CDI and coding processes typically occur independently of each other, even though the two processes are related. This can result in inefficiencies and inaccuracies in both CDI and coding.

### SUMMARY

A computer system implements a concurrent Clinical Document Improvement (CDI) and coding workflow, such as may be used to generate codesets for use in connection with medical bills. The concurrent workflow enables steps in the coding process to be performed close to admission and concurrently with (e.g., interwoven with) the patient visit and steps in the CDI process, thereby increasing the accuracy of the resulting codesets and the efficiency of the codeset generation process. In particular, this concurrent coding workflow may be used to resolve DRG mismatches and enable codesets to be finalized immediately upon discharge of the patient from the hospital.

One aspect of the present invention is directed to a method performed by at least one computer processor executing computer program instructions stored on at least one non-transitory computer readable medium to execute a method. The method includes: generating an initial set of codes based on at least one document; receiving, from a first user, input representing an action to be performed by a second user; storing a record representing the action to be performed by the second user; receiving from the second user, input representing completion of the action to be performed by the second user; updating the record representing the action to be performed by the second user to indicate that the action has been completed; and after the updating, finalizing the initial set of codes.

Other features and advantages of various aspects and embodiments of the present invention will become apparent from the following description and from the claims.

### BRIEF DESCRIPTION OF THE DRAWINGS

FIG. 1 is a flowchart of a method performed by embodiments of the present invention to implement a concurrent Clinical Documentation Improvement (CDI) and coding workflow.

FIG. 2 is a dataflow diagram of a system that performs the method of FIG. 1 according to one embodiment of the present invention.

FIGS. 3A-3C are illustrations of user interfaces according to various embodiments of the present invention.

FIG. 4 is a flowchart of a method according to one embodiment of the present invention.

### DETAILED DESCRIPTION

A computer system implements a concurrent Clinical Document Improvement (CDI) and coding workflow, such as may be used to generate codesets for use in connection with medical bills. The concurrent workflow enables steps in the coding process to be performed close to admission and concurrently with (e.g., interwoven with) the patient visit and steps in the CDI process, thereby increasing the accuracy of the resulting codesets and the efficiency of the codeset generation process. In particular, this concurrent coding workflow may be used to resolve DRG mismatches and enable codesets to be finalized immediately upon discharge of the patient from the hospital.

As mentioned above, existing CDI processes typically review medical documentation in a single workflow. Only after the CDI workflow is complete does billing coding occur. As a result, coding typically does not occur until after the patient has been discharged. Embodiments of the present invention address the problems associated with such post-discharge coding by enabling coding to be performed upon admission and concurrently with CDI.

As further mentioned above, various software exists for facilitating, and even partially automating, both CDI workflows and coding workflows. Such software includes, for example, the ability to prioritize workflows (cases) so that the CDI specialist (in the case of CDI workflows) and the coding specialist (in the case of coding workflows) can work on higher priority cases (workflows) sooner than lower priority cases. The process of prioritizing the CDI specialist's cases is typically independent of the process of prioritizing the coder's cases. As a result, the priorities assigned to the cases for the CDI specialist may differ from the priorities assigned to the same cases for the coding specialist. This can result in inefficiences if, for example, a particular case is prioritized as a high priority for the CDI specialist but as a low priority for the coding specialist.

Furthermore, because of the lack of integration between the CDI and coding workflows in existing systems, workflows in existing systems may be more ineffecient and error prone. For instance, because existing workflows may in some cases not be collaborative and in other cases may not operate concurrently and in real-time, communication between CDI specialists and coding specialists using existing systems is often disjointed.

Embodiments of the present invention address these and other problems with existing systems by providing a computer system and method which enables a collaborative concurrent CDI and coding workflow. This workflow enables seamless communication between CDI specialists and coding specialists throughout the review process. In addition, the manner in which the workflow facilitiates concurrent operation can be leveraged in other contexts. For instance, the manner in which control is exchanged between CDI specialists, coding specialist, or other users of the workflow may generate one or more versions of a set of data that is being used by the workflow and that can be used in operations ancillary to the concurrent workflow. One example of such an operation is an impact analysis, although the different versions of the data can be used in other ways as well.

As a result, embodiments of the present invention enable the coding workflow, which currently is a post-discharge process, to become concurrent with the CDI process, such as while the patient is still in the hospital. This enables embodiments of the present invention to generate a clean and accurate codeset upon or shortly after admission and concurrently in the process than is possible with existing systems, thereby reducing the amount of retrospective coding work required and reducing work required to reconcile codesets with the results of CDI. Codesets evolve as the documentation changes concurrently during the visit. This, in turn, may reduce the number of patients who are categorized as DNFB (discharged, not final billed).

Before describing embodiments of the present invention in more detail, certain terms will used herein will be described.

A diagnosis-related group (DRG) is a patient classification system that standardizes prospective payment to hospitals and encourages cost containment initiatives. In general, a DRG payment covers all charges associated with an inpatient stay from the time of admission to the time of discharge. The DRG includes any services performed by an outside provider. Claims for the inpatient stay are submitted and processed for payment only upon discharge. DRGs categorize patients with respect to diagnosis, treatment, and length of hospital stay. The assignment of a DRG depends on the following variables: principal diagnosis, secondary diagnosis(es), surgical procedures performed, comorbidities and complications, patient's age and sex, and discharge status.

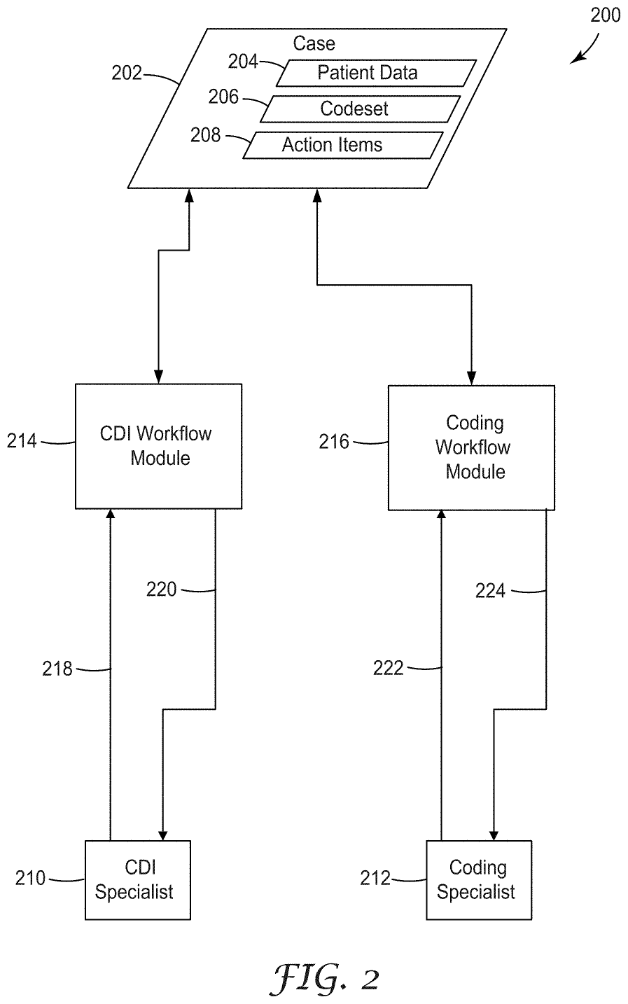

The term “case” refers herein to data relating to a particular patient hospital encounter/visit. For example, referring to FIG. 2, a dataflow diagram is shown of a system 200 according to one embodiment of the present invention. The system 200 includes case data 202 (also referred to herein as a “case”), which includes data 204 representing information about the patient, a codeset 206 associated with that patient's hospital visit, and a set of action items 208 (also referred to herein simply as “actions” or “tasks”) to be performed as part of a concurrent CDI and coding process. The case 202 may include additional data not shown in FIG. 2, such as findings and/or queries.

Although only one case 202 is shown in FIG. 2 for ease of illustration, the system 200 may include any number of cases simultaneously, and any of the techniques disclosed herein may be applied to any such cases. As will be described in more detail below, embodiments of the present invention enable both one or more CDI specialists and one or more coding specialists to collaborate on the same concurrent case, such as the case 202. For ease of explanation, FIG. 2 and the following description will refer to a single CDI specialist 210 and a single coding specialist 212 associated with each case, even though in practice multiple CDI specialists and/or multiple coding specialists may be associated with a case, and in many situations no one CDI specialist communicates exclusively with a specific coding specialist and vice versa.

A single case may include one or more action items, such as action items 208. Each such action item may include one or more properties, such as a name, a type, a due date, and a name or other unique identifier of the person to whom the task is assigned. As will be described in more detail below, a single case may include at least one action item that is assigned to a CDI specialist and at least one action item that is assigned to a coding specialist. As will further be described in more detail below, an action item within a case may be assigned by a CDI specialist to a coding specialist, and an action item within a case may be assigned by a coding specialist to a CDI specialist. These are examples of ways in which embodiments of the present invention may integrate CDI and coding workflows.

The system 200 includes a CDI workflow module 214 which manages one or more CDI workflows. For example, the CDI specialist 210 may provide input 218 of any of a variety of kinds to the CDI workflow module 214, in response to which the CDI workflow module 214 may access (e.g., write to and/or read from) the case 202. The CDI workflow module 214 may provide output 220 to the CDI specialist based on the case 202, such as by displaying some or all of the data in the case 202 to the CDI specialist 210. As described in more detail herein, the CDI workflow module 214 may perform the same functions in connection with a plurality of cases.

The system 200 also includes a coding workflow module 216 which manages one or more coding workflows. For example, the coding specialist 212 may provide input 222 of any of a variety of kinds to the coding workflow module 216, in response to which the coding workflow module 216 may access (e.g., write to and/or read from) the case 202. The coding workflow module 216 may provide output 224 to the coding specialist based on the case 202, such as by displaying some or all of the data in the case 202 to the coding specialist 212. As described in more detail herein, the coding workflow module 216 may perform the same functions in connection with a plurality of cases.

Embodiments of the present invention may, for example, provide output representing the case 202 (such as a visual display of some or all of the data in the case 202) to both the CDI specialist(s) and the coding specialist(s) assigned to (i.e., associated with) the case 202, and may receive input from such CDI specialist(s) and coding specialist(s) and modify the case 202 in response to such input. Any of these functions may be performed while the case's codeset 206 is being modified (e.g., before the case's codeset 206 has been finalized), which may be before the patient associated with the case 202 has been discharged from the hospital.

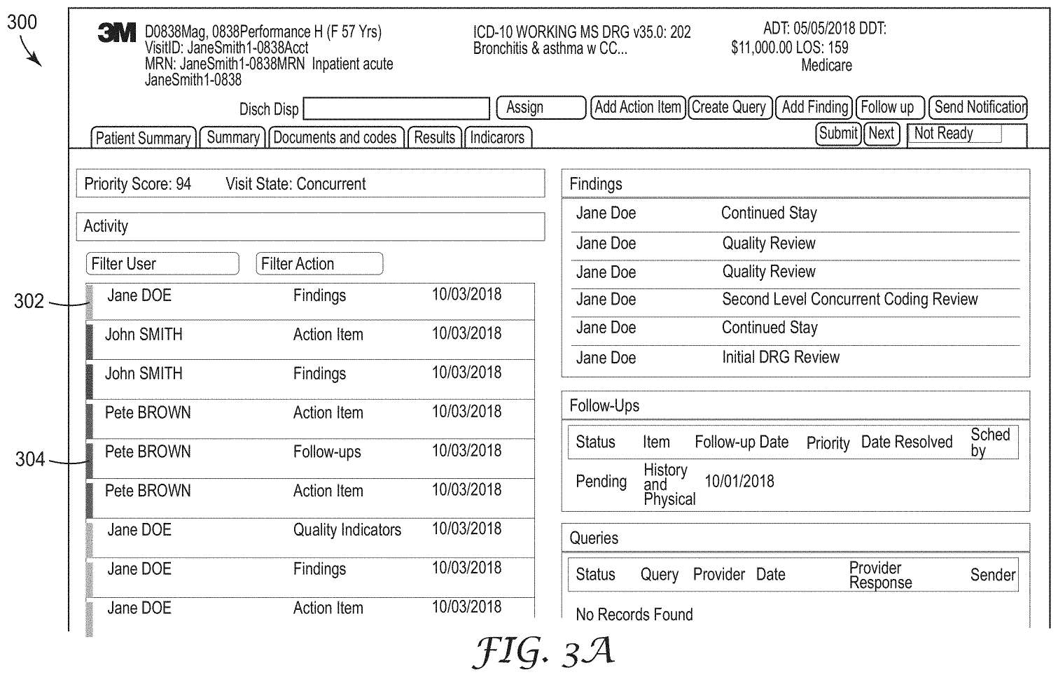

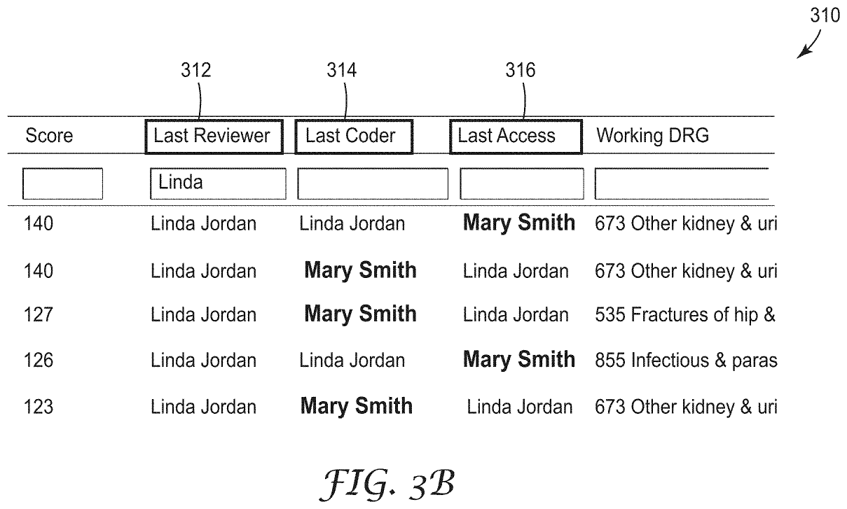

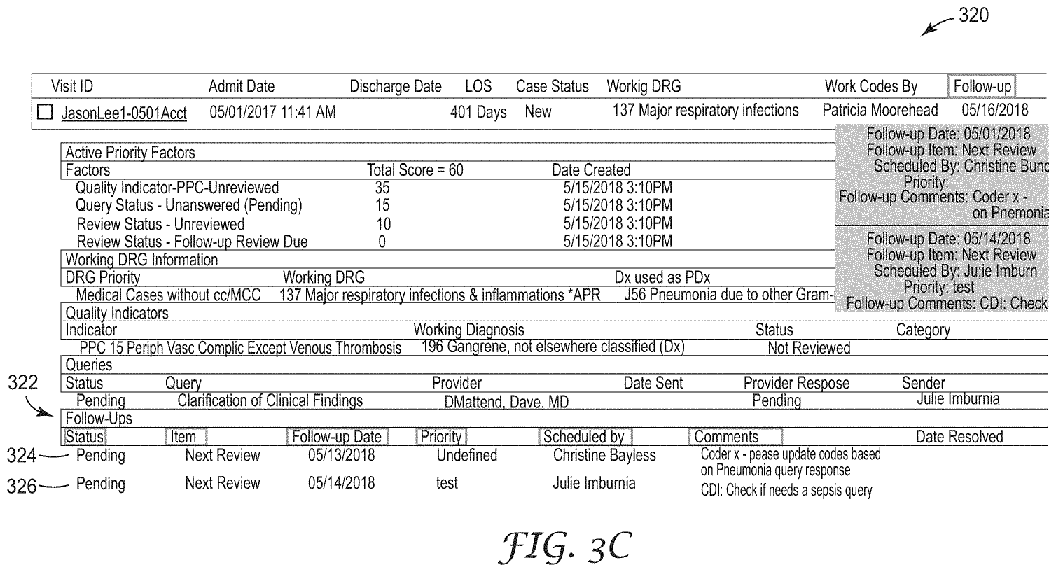

An example of such output is shown in FIG. 3A, which illustrates a user interface 300 displaying a variety of data, such as findings, action items, follow-ups, and quality indicators, in a particular case. The case represented by FIG. 3A includes both data that was created by a CDI specialist and data that was created by a coder. For example, findings 302 were created in the case by a CDI specialist, while follow-ups 304 were created in the case by a coder. The user interface 300 of FIG. 3 may be displayed to a CDI specialist, to a coder, or both. The user interface 300 of FIG. 3, therefore, illustrates how information about a concurrent workflow may be made accessible to both a CDI specialist and a coder while they are working on the same case concurrently.

Referring to FIG. 3B, a user interface 310 is shown which displays a list of users who have accessed a case in a concurrent workflow according to one embodiment of the present invention. Each row in the list represents a particular case. The name of the person in the “Last Reviewer” column 312 of each row is the name of the CDI specialists who last took action in the corresponding case while in a CDI workflow (e.g., who last added a finding or a working DRG). The name of the person in the “Last Coder” column 314 of each row is the name of the coding specialist who last took action in the case while in a coding workflow (e.g., adding a finding or working DRG). The name of the person in the “Last Access” column 316 is the name of the last user (regardless of role) who accessed the case (e.g., added a finding or working DRG).

As will further be described in more detail below, the CDI specialist 210 associated with the case 202 and the coding specialist 212 associated with the case 202 may work on that case 202 concurrently in any of a variety of ways. For example, embodiments of the present invention may provide output 220 and 224 (e.g., visual output as illustrated in FIG. 3A) representing some or all of the case 202 to both the CDI specialist 210 associated with the case 202 and the coding specialist 212 associated with the case 202 simultaneously or contemporaneously. As another example, embodiments of the present invention may receive input 218 from the CDI specialist 210 associated with the case 202, then modify the case 202 in response to the input 218, and then provide output 224 representing some or all of the modified case 202 to the coding specialist 212 associated with the case 202. Such output 224 may be provided to the coding specialist 212 while the case 202 is still in progress, i.e., before the workflow associated with the case 202 (and its associated codeset 206) has been completed.

Similarly, embodiments of the present invention may receive input 222 from the coding specialist 212 associated with the case 202, then modify the case 202 in response to the input 222, and then provide output 220 representing some or all of the modified case 202 to the CDI specialist 210 associated with the case. Such output 220 may be provided to the CDI specialist 210 while the case 202 is still in progress, i.e., before the workflow associated with the case 202 (and its associated codeset 206 and/or activities) has been completed.

The steps described above may be repeated and/or combined together in any of a variety of ways. For example, embodiments of the present invention may modify the case 202 (e.g., the codeset 206 and/or action items 208 associated with the case 202) in response to input 218 received from the CDI specialist 210 associated with the case, then provide output 224 representing some or all of the modified case to the coding specialist 212 associated with the case 202, then receive input 222 from the coding specialist 212, then modify the modified case 202 in response to the input 222 received from the coding specialist 212 to produce a further modified case 202, and then provide output 220 representing some of the further modified case to the CDI specialist 210. These are merely examples of ways in which embodiments of the present invention may provide “concurrent” CDI and coding workflows.

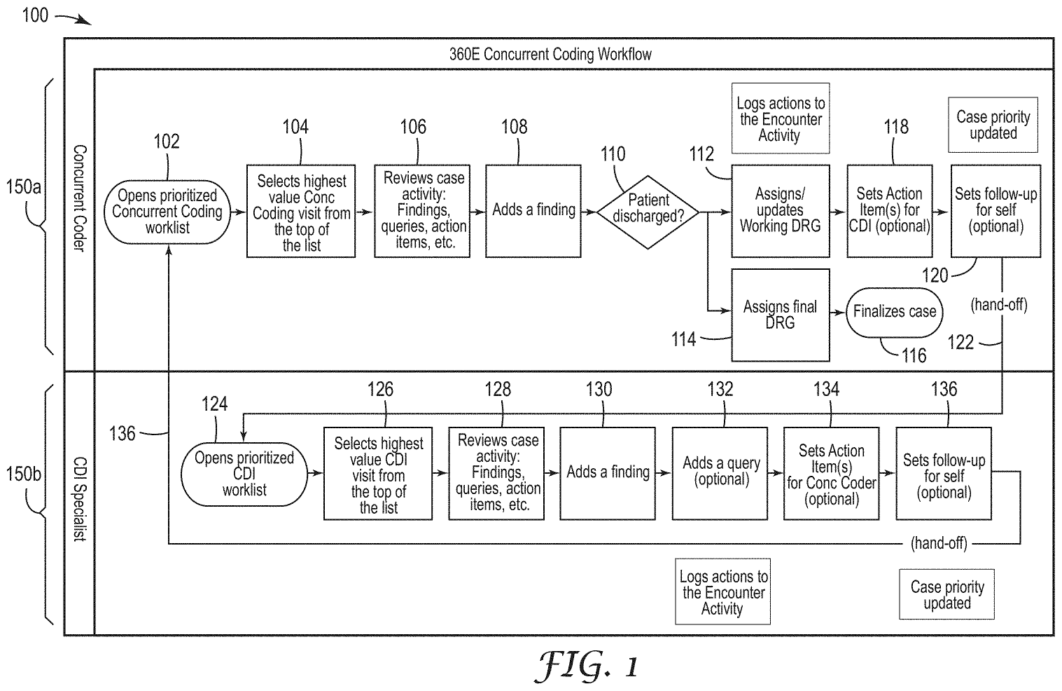

Referring to FIG. 1, a flowchart is shown of a method 100 performed by one embodiment of the present invention. Operations in group 150a are part of a coding workflow, and operations in group 150b are part of a CDI workflow. As will be described in more detail below, the method 100 integrates groups 150a and 150b into a concurrent workflow.

Assume, for purposes of the following description, that the coding specialist 212 has a corresponding worklist, referred to herein as a “coding worklist” or a “concurrent coding worklist.” Also assume, for purposes of the following description, that the CDI specialist 210 has a corresponding worklist, referred to herein as a “CDI worklist.”

Referring to FIG. 3C, a user interface 320 displaying such a worklist is shown according to one embodiment of the present invention. The worklist includes a variety of sections, including Active Priority Factors, Working DRG Information, Quality Indicators, Queries, and Follow-Ups. In particular, and as will be described in more detail below, the Follow-Ups section 322 includes one followup 324 that was created by CDI specialist 210 and is addressed to the coding specialist 212, and includes another followup 326 that was created by the coding specialist 212 and is addressed to the CDI specialist 210.

Before the workflows 150a and 150b begin, data in the case 202 and elsewhere may be generated and stored in any of a variety of ways. For example, while the patient and a physician or other healthcare provider are speaking to each other during the patient's stay, the speech of the patient and/or healthcare provider may be captured and processed using an automatic speech recognition (ASR) engine to produce text representing some or all of that speech. A natural language processing (NLP) engine or a natural language understanding (NLU) engine, which may be integrated with or distinct from the ASR engine, may be applied to the speech and/or corresponding text to identify and generated output representing concepts and other discrete data, such as concepts representing one or more findings, diagnoses, allergies, and prescriptions, among others. The resulting speech and/or discrete data may be stored for example, in plain text, structured text (e.g., XML), an Electronic Health Record (EHR), or any combination thereof. Examples of techniques that may be used to implement such ASR and NLP engines may be found, for example, in U.S. Pat. No. 7,584,103, entitled, “Automated Extraction of Semantic Content and Generation of a Structured Document from Speech, issued on Sep. 1, 2009; and U.S. Pat. No. 7,716,040, entitled, “Verification of Extracted Data,” issued on May 11, 2010.

Referring to FIG. 1, an example method 100 executed by system 200 for implementing a concurrent coding is described. As shown in FIG. 1, the method 100 begins when the system 200 opens the coding specialist 212's coding worklist (FIG. 1, operation 102). The system 200 may, for example, open this worklist in response to input 222 from the the coding specialist 212, such as input representing an instruction to open the worklist. The worklist may already be prioritized (i.e., sorted), in which case the system 200 may display the worklist in its prioritized order, e.g., with the highest priority case on top.

The system 200 selects a particular case from the opened worklist (FIG. 1, operation 104). For example, the system 200 may automatically open the first (e.g., highest priority) case in the opened worklist. As another example, the coding specialist 212 may provide input 222 selecting a particular case from the opened worklist, whether or not that particular case is the highest priority case in the worklist, in response to which the system 200 may open the case selected by the coding specialist. Assume for purposes of example that the selected case is the case 202 shown in FIG. 2. Regardless, the system 200 may provide output 224 (e.g., visual output as illustrated in FIG. 3A) to the coding specialist 212 representing some or all of the data in the selected case 202, which is referred to herein as the “current case.” As a result, the coding specialist 212 may review any and all activity in the current case 202, such as all findings, queries, and action items in the current case 202 (FIG. 1, operation 106). This enables the coding specialist 212 to, for example, review all activity performed by the CDI specialist 210 in the current case 202 so far, to see any queries in the current case 202 and their status, to see any actions that the coding specialist 212 needs to take in the current case, and to see any actions that people other than the coding specialist 212 need to take in the current case 202, even before the current case 202 has been finalized (e.g., before the current case 202's codeset 206 has been finalized and/or before all action items 208 in the current case 202 have been completed).

The system 200 then adds a finding to the current case 202 (FIG. 1, operation 108).

Findings may, for example, be a running list of notes, taken during each review, that indicate pertinent clinical findings in that review. They serve to help anyone who accesses the case 202 to quickly catch up on previous reviews. For example, the coding specialist 212 may provide input 222 to the system 200 specifying a finding, in response to which the system 200 may add the finding specified by the coding specialist's input 222 to the current case 202. The data representing the finding in the current case 202 may include, for example, the name and/or other unique identifier of the coding specialist 212. One benefit of including the coding specialist 212's name and/or other unique identifier in the finding and/or elsewhere in the current case 202 is that other people (such as the CDI specialist 210 associated with the current case 202) may see, when viewing the current case 202, that this particular coding specialist 212 is associated with the current case 202 and therefore is associated with the concurrent workflow illustrated in FIG. 1. Another benefit of including identifying information about the coding specialist 212 in the current case 202 is that this facilitates direct collaboration between the coding specialist 212 and others (e.g., the CDI specialist 210), and facilitates cross-department training and information sharing. That said, it should be understood that, in some implementations, the system 200 does not directly assign a particular coding specialist 212 or a particular CDI specialist 210 to the current case 202. In such implementations, identifying the CDI specialist 210 or the coding specialist 212 (by name or otherwise) may nevertheless facilitate more efficient communication as action items are performed. For instance, if the CDI specialist 210 has questions concerning an action item requested by the coding specialist 212, the CDI specialist 210 may provide input to system 200 indicating a request for additional information from the coding specialist 212. In response, the action item generated by system 200 may be directed to a request for additional information from any user in the coding specialist role as appropriate.

The system 200 then determines, based on information in the current case 202, whether the patient has been discharged (FIG. 1, operation 110). If the system 200 determines that the patient has not been discharged, then the system 200 assigns and/or updates the patient's working DRG (FIG. 1, operation 112). For example, the coding specialist 212 assigned to the current case 202 may provide input 222 representing a new DRG, in response to which the system 200 may create a new DRG associated with the current case 202 (and therefore also associated with the coding specialist 212) in response to the input 222. As another example, the coding specialist 212 assigned to the current case 202 may provide input representing a modification to the current DRG associated with the current case 202 (and therefore also associated with the coding specialist 212), in response to which the system 200 may modify the current DRG based on the input 222. As with all other data in the current case 202, data in such a DRG may be displayed to and otherwise available to the CDI specialist 210 assigned to the current case 202.

If the system 200 determines that the patient has been discharged, then the system 200 assigns a final DRG to the patient in response to input 222 from the coding specialist 212 (FIG. 1, operation 114). “Final” is used as a designation of the DRG after the patient is discharged and no further changes are expected to the case. The system 200 finalizes the case 202 (FIG. 1, operation 116), and the method 100 ends.

If the case 202 is still concurrent, then the system 200 adds an action item for the CDI reviewer 210 to the set of action items 208 (FIG. 1, operation 118). For example, the coding specialist 212 may provide input to the system 200 specifying an action item, in response to which the method 100 may add the action item specified by the coding specialist 212's input to the set of action items 208. The action item may, for example, be assigned to the coding specialist 212 associated with the current case 202 or to the CDI specialist 210 associated with the current case 202. A priority (or weight) may be assigned to the action item, where different action items in the set of action items 208 may have different priorities. Examples of such action items include: evaluate for query, perform CDI review, perform second level CDI review, and perform quality review.

As will be described in more detail below, conversely the CDI specialist 210 associated with the current case 202 may create and assign an action item to the coding specialist 212 associated with the current case. Examples of such action items include: perform a coding review and perform a second level coding review.

When one user assigns an action item to another role in a case (e.g., when the CDI specialist 210 assigns an action item to the coding role, or vice versa), embodiments of the present invention may make that action item immediately visible to and editable by any user assigned to the role in which the action item is assigned. This enables CDI and coding workflows to be integrated and concurrent in a variety of ways. For example, a first user may assign an action item in a case to a second user. Embodiments of the present invention may then display the action item to the second user, such as in a dashboard or otherwise as part of a display of other elements of the same case (e.g., other action items). The second user may edit the action item in any of a variety of ways, such as by adding comments to it and/or marking it as complete. The second user may perform such edits even before the case is complete (e.g., while other action items in the case, such as action items assigned to the first user, remain uncompleted). After the second user has viewed, edited, and/or completed the action item assigned to the second user, the first user may view, edit, and/or complete one or more action items assigned to the first user in the same case. The second user may assign one or more action items to the first user in the case at any time, such as after the first user has assigned the first action item to the second user but before the second user has completed that action item. Embodiments of the present invention may then display the assigned action item to the first user, such as in a dashboard or otherwise as part of a display of other elements of the same case (e.g., other action items).

As these non-exhaustive examples illustrate, the first and second user may concurrently access the case 202 (e.g., by adding, viewing, editing, and/or completing action items in the case 202) in any sequence. As a result, neither user needs to wait until the other user has completed all action items assigned to that user in the case before beginning to work on the tasks assigned to him or her. As a particular example, the coding specialist 212 does not need to wait until the CDI specialist 210 has completed all CDI tasks in the case 202 before beginning to work on coding tasks in the case 202. As will also be described below, implementations of the instant disclosure can ensure data integrity in a variety of ways. For instance, in one implementation, the system 200 generates a new version of the codeset 206 in response to a codeset being changed by users of the system 200 when completing one or more action items generated by the system 200 in response to the system 200 receiving user input. The multiple versions of the codeset 206 can be used by one or more processes ancillary to the workflow for specific purposes.

For instance, multiple versions of codeset 206 can be processed during an impact analysis, which allows administrators and other users of the system 200 to determine the impact that particular users (such as CDI specialist 210 or coding specialist 212) have upon the outcome of the patient's care. For instance, if a CDI specialist 210 notices that the clinical indicators of a diagnosis are present, but does not find that the diagnosis has been documented, the CDI specialist 210 may generate a query asking the healthcare provider to clarify whether there is a diagnosis to be documented. Depending on the resolution of this action (e.g., if a diagnosis was missed), future impact analysis may determine that the CDI specialist 210 had a positive impact on the patient's care. In some implementations, the system 200 may further generate and manage the versions such that the system 200 can access the multiple versions of the codeset 206 during the concurrent workflows. For instance, the system 200 may maintain the version in a database or other storage or archival means for future retrieval.

References herein to “assigning” an action item to a user (also referred to herein as “associating” an action item with a user) may be implemented in any of a variety of ways that are well-known to those having ordinary skill in the art. For example, assigning an action item to a user may include storing, in the action item, data identifying a role of the user that is to perform the action item.

Returning to FIG. 1, after operation 120, the method 100 may hand off the workflow 150a to a workflow 150b of a CDI specialist (FIG. 1, operation 122). As described above, when the method hands off the workflow 150a, the system 200 may generate, e.g., a new version of the codeset 206 for use in workflow 150b. Note that the sequence of operations shown in FIG. 1 is merely an illustrative example, and not a limitation of the present invention. As the description herein makes clear, various operations illustrated in FIG. 1 may be performed in other sequences as part of the concurrent workflow described herein.

The method 100, within the CDI workflow 150b, opens the CDI specialist 210's coding worklist (FIG. 1, operation 124), which may have been produced, at least in part, as the result of one or more of the operations 102-120 previously described in connection with the concurrent coder workflow 150a (FIG. 1, operation 124). The method 100 may, for example, open the CDI specialist 210's coding worklist in response input from the the CDI specialist 210, such as input representing an instruction to open the worklist. The worklist may already be prioritized (i.e., sorted), in which case the method 100 may display the worklist in its prioritized order, e.g., with the highest priority visit on top. In some implementations, the prioritization is for a particular role and is based on one or more action items added to one or more cases. For instance, the system 200 may assign one or more weights to cases (including case 202) in the CDI specialist 210's worklist in response to receiving action items generated by the coding specialist 212 and to be performed by the CDI specialist 210. If, however, the CDI specialist 210 were to generate one or more action items to be performed by the coding specialist 212, those action items may not impact the prioritization of the contents of the CDI specialist 210's worklist because the weights associated with the action items to be performed by the coding specialist 212 are not applied to the CDI specialist 210's workflow, according to particular implementations. That said, one or more actions items generated by the CDI specialist 210 to be performed by the coding specialist 212 may affect the prioritization of the coding specialist 212's worklist according to particular implementations.

The method 100 selects a particular case from the opened worklist (FIG. 1, operation 126). For example, the method 100 may automatically open the first (e.g., highest priority) case in the opened worklist. As another example, the CDI specialist 210 may provide input selecting a particular case from the opened worklist, whether or not that particular case is the highest priority case in the worklist, in response to which the method 100 may open the case selected by the CDI specialist 210. In any event, the method 100 may provide output (e.g., visual output as illustrated in FIG. 3A) representing some or all of the data in the selected case, which is referred to herein as the “current case.” As a result, the CDI specialist 210 may review any and all activity in the current case, such as all findings, queries, and action items in the current case (FIG. 1, operation 128). This enables the CDI specialist 210 to, for example, review all activity performed by the coding specialist 212 and the CDI specialist 210 in the current case 202 so far, to see any queries in the current case 202 and their status, to see any actions that the CDI specialist 210 needs to take in the current case 202, and to see any actions that people other than the CDI specialist 210 need to take in the current case 202, even before the current case 202 has been finalized (e.g., before the current case 202's codeset has been finalized and/or before all activities in the current case 202 have been completed).

The method 100 then adds a finding to the current case 202 (FIG. 1, operation 130). For example, the CDI specialist 210 may provide input to the method 100 specifying a finding, in response to which the method 100 may add the finding specified by the CDI specialist 210's input to the current case 202. The data representing the finding in the current case 202 may include, for example, the role, name, and/or other unique identifier of the CDI specialist 210. One benefit of including the CDI specialist 210's name and/or other unique identifier in the finding and/or elsewhere in the current case 202 is that other people (such as the coding specialist 212 associated with the current case 202) may see, when viewing the current case 202, that this particular CDI specialist 210 is associated with the current case 202 and therefore is associated with the concurrent workflow illustrated in FIG. 1, although as described above there is no requirement that any one CDI specialist 210 or any one coding specialist 212 be assigned to the current case 202.

The method 100 then adds a query to the current case 202 (FIG. 1, operation 132). For example, the CDI specialist 210 may provide input to the method 100 specifying a query, in response to which the method 100 may add the query specified by the CDI specialist 210's input to the current case 202. The CDI specialist 210 may provide queries in any of a variety of situations, such as:
The CDI specialist 210 reviews all documentation in the case 202 and finds clinical indicators of a diagnosis, but does not find that the diagnosis has been documented. In this case, the CDI specialist 210 may send a query to the healthcare provider asking the healthcare provider to clarify whether there is a diagnosis to be documented.
The CDI specialist 201 finds an unspecified diagnosis in the documentation and knows that further specificity in the diagnosis is needed from the healthcare provider. In this case, the CDI specialist 210 may ask the healthcare provider to provide more detail to ensure that the documentation is complete and accurate.

The data representing the query in the current case 202 may include, for example, one or more of the following: (1) a description of the query; (2) the role, name, and/or other unique identifier of the CDI specialist 210 who created the query; and (3) the role, name, and/or other unique identifier of the user (e.g., coding specialist 212) to whom the query is directed. One benefit of including the CDI specialist 210's name and/or other unique identifier in the query is that other people (such as a healthcare provider or the coding specialist 212 associated with the current case) may see, when viewing the query, that the query was created by that particular CDI specialist 210, and can thereby easily contact that CDI specialist 210 to obtain any additional information needed.

The system 200, in response to input from the CDI specialist 210, may then add one or more action items, assigned to the coding specialist 212, to the current case 202 (FIG. 1, operation 134), such as to perform a coding review, or for another concurrent coder to perform a second-level review. An action item may, for example, be a task that is assigned by one role (e.g., coder or CDI specialist) to another role (e.g., coder or CDI specialist), requesting that a new action be taken in the case 202. That said, it should be understood that when, e.g., the system 200 generates a record corresponding to an action item to be performed by the coding specialist 212, the action may be performed by any user of the system that is assigned the coding specialist role. For example, the CDI specialist 210 may provide input corresponding to a request that coding specialist Jane Doe conduct a coding review. In such instances, any coding specialist, including coding specialist Jane Doe, may perform the coding review and mark the action item complete in accordance with implementations of the invention.

The method 100, in response to input from the CDI specialist 210, adds a followup action item, assigned to the CDI specialist 210, to the current case 202 (FIG. 1, operation 136). A followup action item may, for example, be a reminder assigned by a user to himself or herself to check back on the case for new information, such as responses to queries, completed action items, new EHR documents, etc. In operation 136, the method 100 may also close a previously-opened followup or update the followup date of a previously opened followup.

The method 100 then hands control back to the workflow 150a of the concurrent coder 212 (FIG. 1, operation 138). The control may pass back and forth between the concurrent coding workflow 150a and the CDI workflow 150b any number of times. As described above, when control is passed between workflows 150a and 150b, the system 200 may generate versions of the data pertaining to the current case 202. In some implementations, only a subset of the data associated with the case 202 (e.g., codeset 206) is versioned in this way. As a result, the system 200 may generate any number of versions of the data pertaining to the current case 202.

As mentioned above, operations in the method 100 may be performed in sequences other than those shown in FIG. 1. Furthermore, various operations may be omitted from the method 100 of FIG. 1. Furthermore, various operations in the method 100 of FIG. 1 may be repeated any number of times in any sequence.

After the patient has been discharged (FIG. 1, operation 110), the coding specialist 212 assigned to the workflow may complete the workflow, which may include accepting the working codeset 206 associated with the workflow (e.g., the most recent version of the codeset 206), or discarding that working codeset 206 and creating a final codeset (e.g., from a less recent version of the codeset 206), which is associated with the workflow. The method 100, in response to input from the coding specialist 212, may then submit the final DRG for billing (FIG. 1, operation 114). The method 100 then completes and the workflow is updated to mark it as complete (FIG. 1, operation 116).

As described above, cases in a worklist may be assigned corresponding priorities, and the cases in a worklist may be prioritized (sorted) based on those priorities when displayed to a user. The priority of a case may be updated based on changes to data in the workflow during or after any of the operations of the method 100. The worklist that contains the case may then be resorted based on the updated priority of the case. The same is true when the priority of any case in the worklist changes.

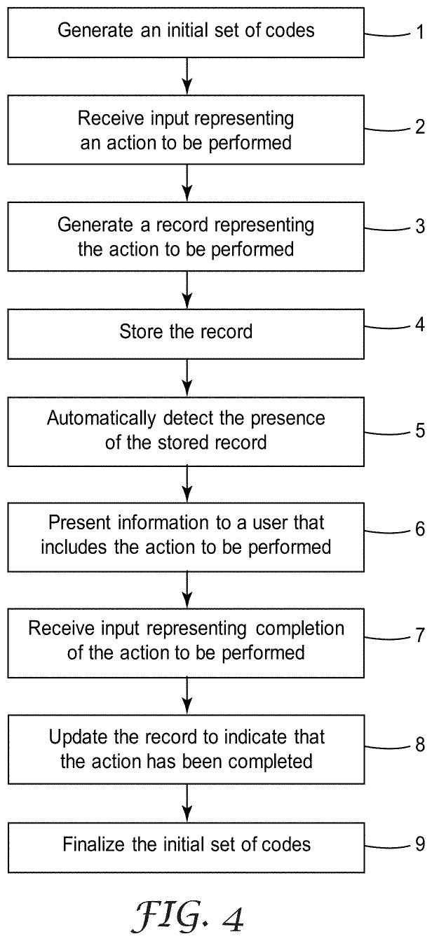

Referring now to FIG. 4, a method performed by system 200 is described to finalize an initial set of codes. The method will be described in connection with one or more specific examples, but it should be understood that the method can be performed by system 200 using, e.g., other queries, actions, users, and user roles not specifically described.

At step 1, the system 200 generates an initial set of codes. Typically, the initial set of codes is based on at least one document in a corpus of documents. The manner in which the system 200 generates the initial set of codes based on the document or documents may vary by implementation. In some implementations, the initial set of codes is generated in response to receiving one or more medical documents in a corpus of medical documents. For instance, the system 200 may receive a document from a healthcare provider as part of a patient/healthcare provider encounter. In some implementations, and as described above, the system 200 generates the initial set of codes by first processing one or more documents using an ASR engine (which may or may not include NLP or NLU subsystems) to identify relevant information to generate the initial set of codes. In some implementations, the system 200 may receive information pertaining to the document after another system processes one or more documents using an ASR engine (which may or may not include NLP or NLU subsystems) and use the received information to generate the initial set of codes. In some implementations, the initial set of codes can be generated from input received by a first user, such as coding specialist 212. Other implementations and combinations thereof are also possible.

At step 2, the system 200 receives input representing an action to be performed. In general, the input is received from a first user in a first role and corresponds to an action to be performed by one or more second users in a second role. For example, the CDI specialist 210 may request that the coding specialist 212 perform a coding review, or for another user in the coding specialist role perform another type of review. As another example, the CDI specialist 210 may request that a healthcare provider review a patient's medical record and to clarify whether there is a diagnosis that remains to be documented. As another example, the coding specialist 212 may request that the CDI specialist 210 follow-up with respect to one or more outstanding issues in a case 202 (e.g., such as requesting that a healthcare provider clarify whether there is a diagnosis to be documented). Other examples of actions to be performed by the CDI specialist 210, the coding specialist 212, and healthcare providers are also possible.

At step 3, the system 200 generates a record representing the action to be performed.

For instance, the system 200 may generate a record corresponding to a request made by coding the specialist 212 asking the CDI specialist 210 to follow-up with one or more outstanding issues in a case 202. In general, the system 200 designates the action as being “open” (i.e., unresolved or otherwise needed to be acted upon). For example, the system 200 can generate the record corresponding to the request with an open status. When the system 200 generates the record, it may assign a weight to the action to be performed, and associate the weight with the record. In general, the system is configured with one or more priority factors that the system 200 uses to determine an appropriate weight to assign the record. These priority factors may differ based on the role that is assigned to perform the requested action item. For instance, if the system 200 generates an action item corresponding to the CDI specialist 210 requesting that a healthcare provider clarify whether a diagnosis remains to be documented, the system 200 may assign the action item a weight of 20 for the healthcare providers. It should be understood that specific values are provided for exemplary purposes only, and the system 200 may use any particular weight values for the purposes outlined herein.

At step 4, the system 200 stores the record. In general, the record is stored in a manner that allows one or more distributed systems (such as one or more systems 200) to access the stored record over a distributed network, such as a wide-area network (“WAN”), or local-area network (“LAN”), or both. For instance, the system 200 can store the record in a database accessible by any system on the distributed network, store the record as a file on storage medium accessible by any system on the distributed network, or upload the record as a file to a cloud-based storage system, or combinations thereof according to particular implementations.

As step 5, the system 200 automatically detects the presence of the stored record. In general, the system 200 can use any number of mechanisms for detecting the presence of the stored record. For instance, the system 200 can automatically detect that a new version of the case 202 (or some portion thereof, e.g., a new version of codeset 206) has been stored when the system 200 executes an operation committing the record to a database, executes a file input/output operation storing the record to a computer-readable storage medium, or executes an operation transmitting the record to a cloud storage system to be stored thereon, according to particular implementations.

At step 6, the system 200 presents information to a user that includes the action to be performed. For instance, if the system 200 generates an action item corresponding to a request from the coding specialist 212 to the CDI specialist 210, the system 200 may open the CDI specialist 210's coding worklist in response to input from the the CDI specialist 210 (e.g., as input representing an instruction to open the worklist provided by the CDI specialist 210). The worklist may be prioritized (i.e., sorted), corresponding to the weights assigned by the system 200 to the various action items in the CDI specialist's workflow. In some implementations, this means in that the system 200 may display the worklist in its prioritized order, e.g., with the highest priority visit on top. Because, as described elsewhere, different weights may be assigned to the various action items created by the system for different user roles, it should be appreciated that different workflows are prioritized in different ways by the system. For example, the CDI specialist 210's workflow may be prioritized differently than the coding specialist 212's workflow on the basis of the different weights assigned to tasks for the the CDI specialist 210's role and the coding specialist 212's role, respectively.

At step 7, the system 200 receives input representing completion of the action to be performed. Continuing with the example of step 3, above, where the CDI specialist 210 requested that a healthcare provider clarify whether a diagnosis remains to be documented, the healthcare provider can respond to the request by providing an updated diagnosis if the medical records support such an action. In other situations, the healthcare provider may respond to the request by indicating that no new diagnosis is appropriate under the circumstances.

At step 8, the system 200 updates the record to indicate the action has been completed. For instance, after the healthcare provider responds to the request and provided input to the system 200 representing completion of that tasks, the system 200 may generate a new version of the case 202 (or a portion thereof, such as codeset 206) indicating the action has been performed. In some implementations, the system 200 can change the status of the action item from “open” to “resolved” to indicate that an action has been completed. By changing the status of the action to be performed from open to resolved, the action would not be displayed to subsequent users in the role for which the action was to generated. For example, if an action was generated by a coding specialist 212 for a user in the CDI specialist role, upon completion, the action would be removed from a In so doing the system 200 may perform one or more additional tasks according to particular implementations.

In one example, the system 200 removes the weight associated with the performed action from the case 202, which may modify the prioritization of the cases presented to the CDI specialist 210 when the system 200 presents the CDI specialist 210's workflow. For instance, because the system 200 has removed the weight associated with the completed action, the system 200 would present the case 202 lower in priority order to the CDI specialist 210. In another example, after removing the weight associated with the completed action item, the system 200 may add one or more weights to the case 202 on the basis of the action being completed which may modify the prioritization of the case 202 for one or more user roles in the system 200.

For instance, after the healthcare provider responds to the query from the CDI specialist 210, the system 200 may add weights to both the case 202 for both the CDI specialist 210 an the coding specialist 212. In some implementations, the added weights may differ such that the case 202 is presented in different priority order for the CDI specialist 210 and the the coding specialist 212. For example, the system 200 may assign the response of the healthcare provider a weight of 20 in the case 202 for the CDI specialist 210 and assign a weight of 10 in the case 202 for the coding specialist 212. In this particular example, the weight may be higher for the CDI specialist 210 because users in the CDI specialist's role may be responsible for resolving the query and updating the information associated with case 202, such as medical chart information. Meanwhile, the weight assigned by the system 200 to users in the coding specialist 212's role may reflect general activity in case 202, but no specific action to be performed by the coding specialist 212. As a result, the case 202 would appear higher in priority for the CDI specialist 210 than the coding specialist 212 in the CDI specialist 210's and coding specialist 212's respective workflows when such workflows are presented by the system 200.

As step 9, the system 200 finalizes the initial set of codes. For instance, the system 200 can determine that the patient associated with the case 202 has been discharged. In some implementations, the system 200 determines the patient has been discharged upon receipt of user input from the CDI specialist 210, the coding specialist 212, a healthcare provider, or combinations thereof. If the system 200 determines that the patient has been discharged, the system 200 may access the most current version of case 202 and/or codeset 206 and use the most current codeset as the final set of codes.

Embodiments of the present invention have a variety of advantages, some of which have been described above. For example, embodiments of the present invention enable coding and CDI specialists to communicate seamlessly throughout an integrated and concurrent coding workflow. Such a concurrent coding workflow may be used to eliminate or significantly reduce the need to reconcile mismatched codesets generated by the coding specialist and CDI specialist, because embodiments of the present invention enable both of those specialists to communicate with each other and see each other's work while a common codeset is being developed.

In particular, embodiments of the present invention provide coders with tools that enable them to review cases and to create working codesets while the patient is still in-house. This provides significant benefits because, for example, it is much simpler and faster for the coder to obtain accurate information about the patient from healthcare providers while they are actively caring for the patient. Once the patient has been discharged, it becomes much more difficult to obtain accurate information about the patient from healthcare providers. Furthermore, the concurrent workflows enabled by embodiments of the present invention allow the coding process to be completed more quickly, thereby reducing the frequency of patients who are categorized as DNFB (discharged, not final billed).

Embodiments of the present invention enable the highest value cases to be prioritized separately for both coding specialists and CDI specialists, so that both categories of user can target the highest-priority cases first. This may result in a reduction in denials, improvement in documentation accuracy, improvement in accuracy of the codeset, and an increase in quality scores.

One aspect of the present invention is directed to a method performed by at least one computer processor executing computer program instructions stored on at least one non-transitory computer readable medium to execute a method. The method includes generating an initial set of codes based on at least one document; receiving, from a first user, input representing an action to be performed by a second user; storing a record representing the action to be performed by the second user; receiving from the second user, input representing completion of the action to be performed by the second user; updating the record representing the action to be performed by the second user to indicate that the action has been completed; and after the updating, finalizing the initial set of codes.

Before generating the initial set of codes, the method may generate the document by applying automatic speech recognition to a signal representing speech. The method may notify the second user of the record representing the action to be performed by the second user.

The method may further include: receiving, from the second user, input representing an action to be performed by the first user; storing a record representing the action to be performed by the first user; and receiving from the first user, input representing completion of the action to be performed by the first user. The method may further include: displaying, to the second user, output representing the first action and the second action. The output representing the first action and the second action may be displayed to the second user before finalizing the initial set of codes. The method may further include: displaying, to the first user, output representing the first action and the second action. The output representing the first action and the second action may be displayed to the first user before finalizing the initial set of codes.

The method may further include: receiving, from the first user, input indicating a modification to the initial set of codes; and modifying the initial set of codes to produce a modified set of codes based on the input indicating the modification to the initial set of codes. The method may further include: displaying, to the first user, the modified set of codes. The method may further include: displaying, to the second user, the modified set of codes.

It is to be understood that although the invention has been described above in terms of particular embodiments, the foregoing embodiments are provided as illustrative only, and do not limit or define the scope of the invention. Various other embodiments, including but not limited to the following, are also within the scope of the claims. For example, elements and components described herein may be further divided into additional components or joined together to form fewer components for performing the same functions.

Any of the functions disclosed herein may be implemented using means for performing those functions. Such means include, but are not limited to, any of the components disclosed herein, such as the computer-related components described below.

The techniques described above may be implemented, for example, in hardware, one or more computer programs tangibly stored on one or more computer-readable media, firmware, or any combination thereof. The techniques described above may be implemented in one or more computer programs executing on (or executable by) a programmable computer including any combination of any number of the following: a processor, a storage medium readable and/or writable by the processor (including, for example, volatile and non-volatile memory and/or storage elements), an input device, and an output device. Program code may be applied to input entered using the input device to perform the functions described and to generate output using the output device.

Any of the functions disclosed herein as being performed in connection with a first user (e.g., a coding specialist) may be performed by a first computing device, such as any one or more of the following in any combination: a desktop computer, a laptop computer, a tablet computer, and a smartphone. Such a computer may perform operations and manipulate data as disclosed herein either directly on a local computer, remotely over a network in communication with a server or other remote computer, or a combination thereof.

Similarly, any of the functions disclosed herein as being performed in connection with a second user (e.g., a CDI specialist) may be performed by a second computing device, such as any one or more of the following in any combination: a desktop computer, a laptop computer, a tablet computer, and a smartphone. Such a computer may perform operations and manipulate data as disclosed herein either directly on a local computer, remotely over a network in communication with a server or other remote computer, or a combination thereof.

Such a first and second computing device may communicate and interoperate with a common server or other remote computer to perform functions disclosed herein. For example, when a first user (e.g., coding specialist) uses a first computing device to update a workflow, the first computing device may communicate with a remote computer, which may update a remote instance of the workflow. The remote computer may then transmit or otherwise make available the updated remote instance of the workflow available to a second user (e.g., CDI specialist) for viewing and/or editing, such as by providing the updated workflow to a second computing device accessible to the second user. Conversely, when the second user (e.g., CDI specialist) uses the second computing device to update the workflow, the second computing device may communicate with the same remote computer, which may further update the remote instance of the workflow. The remote computer may then transmit or otherwise make available the further updated remote instance of the workflow available to the first user for viewing and/or editing, such as by providing the further updated workflow to the first computing device accessible to the first user.

Embodiments of the present invention include features which are only possible and/or feasible to implement with the use of one or more computers, computer processors, and/or other elements of a computer system. Such features are either impossible or impractical to implement mentally and/or manually. For example, embodiments of the present invention enable a CDI specialist and a coding specialist to access and edit a workflow, and data contained within the workflow, concurrently, such as using separate computers connected via a network. These are functions which are inherently computer-implemented, and which could not be performed manually or mentally by a human.

Any claims herein which affirmatively require a computer, a processor, a memory, or similar computer-related elements, are intended to require such elements, and should not be interpreted as if such elements are not present in or required by such claims. Such claims are not intended, and should not be interpreted, to cover methods and/or systems which lack the recited computer-related elements. For example, any method claim herein which recites that the claimed method is performed by a computer, a processor, a memory, and/or similar computer-related element, is intended to, and should only be interpreted to, encompass methods which are performed by the recited computer-related element(s). Such a method claim should not be interpreted, for example, to encompass a method that is performed mentally or by hand (e.g., using pencil and paper). Similarly, any product claim herein which recites that the claimed product includes a computer, a processor, a memory, and/or similar computer-related element, is intended to, and should only be interpreted to, encompass products which include the recited computer-related element(s). Such a product claim should not be interpreted, for example, to encompass a product that does not include the recited computer-related element(s).

Each computer program within the scope of the claims below may be implemented in any programming language, such as assembly language, machine language, a high-level procedural programming language, or an object-oriented programming language. The programming language may, for example, be a compiled or interpreted programming language.

Each such computer program may be implemented in a computer program product tangibly embodied in a machine-readable storage device for execution by a computer processor. Method steps of the invention may be performed by one or more computer processors executing a program tangibly embodied on a computer-readable medium to perform functions of the invention by operating on input and generating output. Suitable processors include, by way of example, both general and special purpose microprocessors. Generally, the processor receives (reads) instructions and data from a memory (such as a read-only memory and/or a random-access memory) and writes (stores) instructions and data to the memory. Storage devices suitable for tangibly embodying computer program instructions and data include, for example, all forms of non-volatile memory, such as semiconductor memory devices, including EPROM, EEPROM, and flash memory devices; magnetic disks such as internal hard disks and removable disks; magneto-optical disks; and CD-ROMs. Any of the foregoing may be supplemented by, or incorporated in, specially-designed ASICs (application-specific integrated circuits) or FPGAs (Field-Programmable Gate Arrays). A computer can generally also receive (read) programs and data from, and write (store) programs and data to, a non-transitory computer-readable storage medium such as an internal disk (not shown) or a removable disk. These elements will also be found in a conventional desktop or workstation computer as well as other computers suitable for executing computer programs implementing the methods described herein, which may be used in conjunction with any digital print engine or marking engine, display monitor, or other raster output device capable of producing color or gray scale pixels on paper, film, display screen, or other output medium.

Any data disclosed herein may be implemented, for example, in one or more data structures tangibly stored on a non-transitory computer-readable medium. Embodiments of the invention may store such data in such data structure(s) and read such data from such data structure(s).

1. A method performed by at least one computer processor executing computer program instructions stored on at least one non-transitory computer readable medium to execute a method, the method comprising:
generating an initial set of codes based on at least one document;
receiving, from a first user assigned to a first role, input representing a first action to be performed by one or more users assigned to a second role;
generating a record representing the first action to be performed by the one or more users in the second role, wherein the record is assigned an opened status and includes a weight associated with the action to be performed;
storing the record representing the first action to be performed by the one or more users in the second role;
receiving from a second user assigned to the second role, input representing completion of the first action to be performed by the one or more users in the second role;
updating, in real-time and over a distributed network, the record representing the first action to be performed by the one or more users in the second role to indicate that the first action has been completed, which includes marking the first action as resolved, removing the weight associated with the first action to generate an updated record, and storing the updated record;
after the updating, finalizing the initial set of codes, wherein
input from the first user and input from the second user are received over the distributed network.

2. The method of claim 1, further comprising:
receiving, from the first user, input representing a finding, and
displaying, to the one or more users in the second role, output representing the finding.

3. The method of claim 1, further comprising:
notifying the one or more users in the second role of the record representing the first action to be performed by the one or more users in the second role.

4. The method of claim 1, further comprising
receiving, from the second user, input representing a second action to be performed by the one or more users in the first role;
generating a record representing the second action to be performed by the one or more users in the first role, wherein the record is assigned an opened status and includes a weight associated with the second action to be performed;
storing a record representing the second action to be performed by the one or more users in the first role;
receiving from the first user assigned to the first role, input representing completion of the second action to be performed by the one or more users in the first role; and
updating, in real-time and over a distributed network, the record representing the second action to be performed by the one or more users in the first role to indicate that the second action has been completed, which includes marking the second action as resolved, removing the weight associated with the second action to generate an updated record for the second action, and storing the updated record.

5. The method of claim 4, further comprising:
displaying, to the second user, output representing the first action and the second action.

6. The method of claim 5, wherein the displaying, to the second user, the output representing the first action and the second action is performed before finalizing the initial set of codes.

7. The method of claim 4, further comprising:
displaying, to the first user, output representing the first action and the second action.

8. The method of claim 7, wherein the displaying, to the first user, the output representing the first action and the second action is performed before finalizing the initial set of codes.

9. The method of claim 1, further comprising:
receiving, from the first user, input indicating a modification to the initial set of codes; and
modifying the initial set of codes to produce a modified set of codes based on the input indicating the modification to the initial set of codes.

10. The method of claim 9, further comprising:
displaying, to the first user, the modified set of codes.

11. The method of claim 9, further comprising:
displaying, to the second user, the modified set of codes.

12. The method of claim 1, further comprising:
prioritizing, using one or more assigned weights stored in association with one or more respective actions for the one or more users in the second role, the one or more actions to be performed by the one or more users in the second role; and
displaying the one or more actions in a prioritized order to the second user.

13. A system including at least one non-transitory computer readable medium comprising computer program instructions which, when executed by at least one computer processor, perform a method, the method comprising:
generating an initial set of codes based on at least one document;
receiving, from a first user assigned to a first role, input representing a first action to be performed by one or more users assigned to a second role;
generating a record representing the first action to be performed by the one or more users in the second role, wherein the record is assigned an opened status and includes a weight associated with the action to be performed;
storing the record representing the first action to be performed by the one or more users in the second role;
receiving from a second user assigned to the second role, input representing completion of the first action to be performed by the one or more users in the second role;
updating, in real-time and over a distributed network, the record representing the first action to be performed by the one or more users in the second role to indicate that the first action has been completed, which includes marking the first action as resolved, removing the weight associated with the first action to generate an updated record, and storing the updated record;
after the updating, finalizing the initial set of codes, wherein
input from the first user and input from the second user are received over the distributed network.

14. The system of claim 13, wherein the method further comprises:
receiving, from the first user, input representing a finding, and
displaying, to the second user on a display device coupled to the system, output representing the finding.

15. The system of claim 13, wherein the method further comprises:
notifying the second user of the record representing the action to be performed by the second user.

16. The system of claim 13, wherein the method further comprises:
receiving, from the second user, input representing a second action to be performed by the one or more users in the first role;
generating a record representing the second action to be performed by the one or more users in the first role, wherein the record is assigned an opened status and includes a weight associated with the second action to be performed;
storing a record representing the second action to be performed by the one or more users in the first role;
receiving from the first user assigned to the first role, input representing completion of the second action to be performed by the one or more users in the first role; and
updating, in real-time and over a distributed network, the record representing the second action to be performed by the one or more users in the first role to indicate that the second action has been completed, which includes marking the second action as resolved, removing the weight associated with the second action to generate an updated record for the second action, and storing the updated record.

17. The system of claim 16, wherein the method further comprises:
displaying, to the second user on a display device coupled to the system, output representing the first action and the second action.

18. The system of claim 17, wherein the displaying, to the second user on a display device coupled to the system, the output representing the first action and the second action is performed before finalizing the initial set of codes.

19. The system of claim 13, wherein the method further comprises:
receiving, from the first user, input indicating a modification to the initial set of codes; and
modifying the initial set of codes to produce a modified set of codes based on the input indicating the modification to the initial set of codes.

20. The system of claim 13, wherein the method further comprises:
prioritizing, using one or more assigned weights stored in association with one or more respective actions for the one or more users in the second role, the one or more actions to be performed by the one or more users in the second role; and
displaying, on a display device coupled to the system, the one or more actions in a prioritized order to the second user.
